(19) United States
(12) Patent Application Publication
(10) Pub. No.: US 2020/0171034 A1
(43) Pub. Date: Jun. 4, 2020

(54) Methods Of Treating Follicular Lymphoma

(71) Applicant: Janssen Biotech, Inc., Horsham, PA (US)

(72) Inventor: Sriram Balasubramanian, Spring House, PA (US)

(21) Appl. No.: 16/696,092

(22) Filed: Nov. 26, 2019

Related U.S. Application Data
(60) Provisional application No. 62/773,678, filed on Nov. 30, 2018.

(51) Int. Cl. A61K 31/519; A61P 35/00

(52) U.S. Cl. CPC A61K 31/519; A61P 35/00

(57) ABSTRACT

Provided herein are methods of treating follicular lymphoma (FL) and gene mutations that can be used to predict a subject's nonresponsiveness to treatment of follicular lymphoma with ibrutinib.

### CROSS REFERENCE TO RELATED APPLICATIONS

This application claims priority to U.S. Provisional Application No. 62/773,678, filed Nov. 30, 2018, the disclosure of which is hereby incorporated by reference in its entirety.

### TECHNICAL FIELD

Provided herein are methods of treating follicular lymphoma (FL) and gene mutations that can be used to predict a subject's nonresponsiveness to treatment of follicular lymphoma with ibrutinib.

### BACKGROUND

The genetic landscape of follicular lymphoma is complex. In addition to the hallmark t(14;18) translocation resulting in BCL2 overexpression, molecular genetic studies have also identified recurrent somatic mutations in a number of genes. Such mutations may reduce a subject's responsiveness to therapy.

### SUMMARY

Provided herein are methods of treating follicular lymphoma (FL) in a subject, the methods comprising administering to the subject a therapeutically effective amount of ibrutinib to thereby treat the FL, wherein the subject does not have one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1.

Also provided are methods of predicting a likelihood of nonresponsiveness to ibrutinib in a subject having follicular lymphoma, the method comprising analyzing a sample from the subject for one or more of the mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1, wherein one or more of the mutations in the one or more genes is indicative of nonresponsiveness to ibrutinib.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 illustrates the number of mutated genes in the DAWN study patients with responder data (N=83).

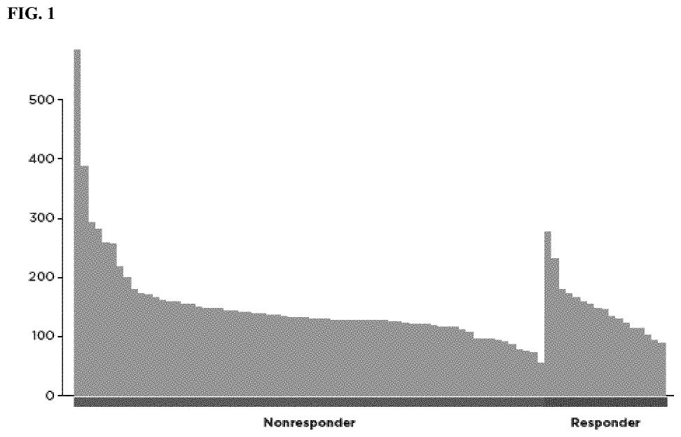

FIG. 2 illustrates a heatmap of genes mutated in >10% of samples (75 genes) from the DAWN study.

FIG. 3 illustrates a heatmap of ranked nonresponder gene mutations from the DAWN study.

FIG. 4 illustrates the mean ORR of predicted responders based on cross-validation studies.

FIG. 5 is an exemplary plot of somatic mutations in the ATP6AP1 gene in DAWN patients.

FIG. 6 is an exemplary plot of somatic mutations in the EP400 gene in DAWN patients.

FIG. 7 is an exemplary plot of somatic mutations in the ARID1A gene in DAWN patients.

FIG. 8 is an exemplary plot of somatic mutations in the SOCS1 gene in DAWN patients.

FIG. 9 is an exemplary plot of somatic mutations in the TBL1XR1 gene in DAWN patients.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values is used to indicate that the recited values may vary by up to as much as 10% from the listed value. Thus, the term “about” is used to encompass variations of +10% or less, variations of 5% or less, variations of 1% or less, variations of 0.5% or less, or variations of +0.1% or less from the specified value.

The term “comprising” is intended to include examples encompassed by the terms “consisting essentially of” and “consisting of”; similarly, the term “consisting essentially of” is intended to include examples encompassed by the term “consisting of.”

Ibrutinib, a first-in-class, oral, covalent inhibitor of Bruton's tyrosine kinase (BTK), approved for several B-cell malignancies in the United States and other countries, disrupts signaling pathways essential for the adhesion, proliferation, homing, and survival of malignant B cells.

“Treat,” “treatment,” and like terms include reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the follicular lymphoma. Treatment includes complete response and partial response to the administered agent (ibrutinib). Treatment also includes prolonging survival as compared to the expected survival of a subject not receiving treatment.

As used herein, the phrase “therapeutically effective amount” refers to an amount of the ibrutinib, as described herein, effective to achieve a particular biological or therapeutic result such as, but not limited to, biological or therapeutic results disclosed, described, or exemplified herein. The therapeutically effective amount may vary according to factors such as the disease state, age, sex, and weight of the individual, and the ability of the composition to cause a desired response in a subject. Exemplary indicators of a therapeutically effective amount include, for example, improved well-being of the patient, reduction of a tumor burden, arrested or slowed growth of the follicular lymphoma, and/or absence of metastasis of follicular lymphoma cells to other locations in the body.

The term “subject” as used herein is intended to mean humans. “Subject” and “patient” are used interchangeably herein.

The following abbreviations are used herein: Bruton's tyrosine kinase (BTK); relapsed or refractory (R/R); overall response rate (ORR); overall survival (OS); follicular lymphoma (FL); complete response (CR); and partial response (PR).

### Methods of Treating Follicular Lymphoma and Uses

Provided herein are methods of treating follicular lymphoma (FL) in a subject, the methods comprising:

administering to the subject a therapeutically effective amount of ibrutinib to thereby treat the FL, wherein the subject does not have one or more mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1.

The mutations provided in Table 2 in one or more of AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1 are associated with nonresponsiveness to ibrutinib treatment, as disclosed herein. Thus, the methods comprise administering to the subject a therapeutically effective amount of ibrutinib to thereby treat the FL, wherein the subject does not have one or more mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1. The methods can be performed on subjects not having one or more mutations as defined in Table 2 in 1, 2, 3, 4, 5, 6, 7, 8, 9, 10, 11, 12, 13, 14, 15, 16, or all 17 of AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1 as provided in Table 2 and various combinations thereof.

Also disclosed are methods of treating follicular lymphoma (FL) in a subject, the methods comprising:

to a subject having FL and not having one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1 administering a therapeutically effective amount of ibrutinib to thereby treat the FL.

Also provided are methods of treating follicular lymphoma (FL) in a subject not having one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1, the methods comprising administering to the subject a therapeutically effective amount of ibrutinib to thereby treat the FL.

The therapeutically effective amount of ibrutinib can comprise from about 420 mg to about 840 mg. For example, the therapeutically effective amount of ibrutinib can comprise about 420 mg, 440 mg, 460 mg, 480 mg, 500 mg, 520 mg, 540 mg, 560 mg, 580 mg, 600 mg, 620 mg, 640 mg, 660 mg, 680 mg, 700 mg, 720 mg, 740 mg, 760 mg, 780 mg, 800 mg, 820 mg, or 840 mg. In some embodiments, the therapeutically effective amount of ibrutinib is 560 mg.

In some embodiments, the FL is relapsed/refractory (R/R) FL.

Suitable subjects for treatment include those who, prior to the administering:
had a diagnosis of grade 1, 2, or 3a nontransformed FL;
had been treated with >2 prior lines of therapy;
was R/R to a last prior line of therapy with an anti-CD20 monoclonal antibody-containing chemoimmunotherapy regimen; or
any combination thereof.

In some embodiments, the subject can have a partial response. In some embodiments, the subject can have a complete response.

Further provided is the use of ibrutinib in the manufacture of a medicament for the treatment of follicular lymphoma (FL) in a subject not having one or more mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1.

Also provided is ibrutinib for use in the treatment of follicular lymphoma (FL) in a subject not having one or more mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1.

### Methods of Predicting a Likelihood of Nonresponsiveness to Ibrutinib in a Subject Having Follicular Lymphoma

Provided are methods of predicting a likelihood of nonresponsiveness to ibrutinib in a subject having follicular lymphoma, the methods comprising: analyzing a sample from the subject for one or more mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1, wherein a mutation in the one or more genes is indicative of nonresponsiveness to ibrutinib.

The mutations provided in Table 2 in one or more of AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1 are indicative of nonresponsiveness to ibrutinib treatment, as disclosed herein. A mutation in 1, 2, 3, 4, 5, 6, 7, 8, 9, 10, 11, 12, 13, 14, 15, 16, or all 17 of AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1 as provided in Table 2 and various combinations thereof can be indicative of nonresponsiveness to ibrutinib treatment.

In some embodiments, the methods comprise analyzing a sample from the subject for one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1, wherein a lack of the one or more mutations in the one or more genes is indicative of responsiveness to the ibrutinib.

In some embodiments, methods of predicting a likelihood of nonresponsiveness to ibrutinib in a subject having follicular lymphoma is combined with a subsequent treatment of the follicular lymphoma. Thus, provided are methods of treating follicular lymphoma (FL) in a subject, the methods comprising:

analyzing a sample from the subject for one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1, wherein the one or more mutations in the one or more genes is indicative of nonresponsiveness to ibrutinib

and administering a therapeutically effective amount of ibrutinib to thereby treat the FL if the subject does not have the one or more mutations in the one or more genes.

Suitable samples from the subject include any biological sample that contains the gene of interest including, but not limited to, whole blood samples and tumor biopsy samples.

### EXAMPLES

The following examples are provided to further describe some of the embodiments disclosed herein. The examples are intended to illustrate, not to limit, the disclosed embodiments.

### Identification of a Genetic Signature Enriching for Response to Ibrutinib in Relapsed/Refractory Follicular Lymphoma (FL)

The DAWN study (NCT01779791) evaluated the efficacy and safety of ibrutinib monotherapy in patients with relapsed/refractory (R/R) follicular lymphoma (FL). The overall response rate (ORR) for ibrutinib was 20.9% (95% confidence interval [CI], 13.7-29.7), not meeting the primary end point. However, responders experienced a long duration of response (median 19.4 months). A genetic investigation was performed on samples from the DAWN study to determine whether somatic mutations could be used to identify FL patients who will respond, or not respond, to ibrutinib.

### Study Design and Patients

Detailed methodology for the DAWN trial is published in Gopal A K, et al. J Clin Oncol. 2018; 36:2405-2412. Briefly, DAWN was a multicenter, single-arm, phase 2 study of ibrutinib (560 mg once daily) in patients aged >18 years with a diagnosis of grade 1, 2, or 3a nontransformed FL who had been treated with >2 prior lines of therapy, and were R/R to their last prior line of therapy with an anti-CD20 monoclonal antibody-containing chemoimmunotherapy regimen. The primary end point was overall response rate (ORR=complete response [CR]+partial response [PR]), assessed by an independent review committee using the International Working Group Revised Response Criteria for Malignant Lymphoma.

Whole exome sequencing was performed on 88 formalin-fixed, paraffin-embedded tumor samples (LabCorp, Burlington, N.C.) from responders or nonresponders following ibrutinib treatment. Multiple filters were applied to rule out potential germline variants, and a custom panel of 1216 genes known to be involved in cancer was used for further analysis. Variants enriched in responders or nonresponders were identified using Fisher's exact test. Variants were marked as “deleterious” based on meta-analytic support vector machine (metaSVM) annotations in the database for nonsynonymous single nucleotide polymorphisms functional predictions (dbNSFP). Classifiers were built with variable numbers of genes ranked with a greedy algorithm that selected genes that would, at each iteration, allow the removal of the greatest number of nonresponders from the patient pool, while severely penalizing the removal of responders. Classification results were first assessed with 10-fold cross-validation within the DAWN dataset, subsequently (See Bartlett N L, et al. Blood. 2018; 131:182-190).

### Sample Collection and Processing

Whole blood samples and tumor biopsy samples were collected and whole blood and plasma fractions were used for gene analysis.

Exome data were generated from FFPE samples of 88 subjects with FL, each from a different subject. Eighty-three of these subjects were indicated as either “responder” (CR+PR) or “nonresponder” (SD+PD) after ibrutinib treatment.

### Exome Sequencing

Whole-exome data was generated using Nimblegen kits and sequencing libraries were made using KAPA construction kits. Sequencing was performed using the Illumina HiSeq2500 platform with a goal of 100× coverage for each sample.

### Variant Calling/Annotation

A total of 88 FFPE FL samples had full exome sequencing performed and were analyzed first by LabCorp. Results of LabCorp analyses were examined by generating a variant allele frequency (VAF) histogram to qualitatively assess (a) the degree to which somatic vs. germline variants were present in the data and (b) whether low VAF variants were properly represented in the set of calls. Since large peaks were seen near VAF=0.5 and VAF=1.0, it was inferred that a large proportion of the variants were likely to be heterozygous or homozygous germline variants; as very few variants were seen at the low end of the VAF histogram, it was determined that a procedure should be used to specifically enrich for the low VAF variants.

To correct for the potential issues seen in the LabCorp data, an in-house exome analysis pipeline was run on DNAnexus using raw FASTQ sequence data files. Quality was assessed using FastQC 1.0.0, sequences were aligned to the hs37d5 genome build using the BWA-MEM algorithm in BWA Software Package 0.5.9, alignments were recalibrated with the GATK 3.5 Exome Pipeline, and variants were annotated with MuTect 1.1.7, SnpEff 4.2 (using the GRCh37.75 database), and GEMINI 0.20.0 (modified by using non-TCGA gnomAD and ExAC references). Non-synonymous coding variants (defined in R as is_coding=“1” & impact!=“synonymous_variant”) were filtered to reduce the likelihood of incorporating sequencing artifacts and germline variants into the association analysis. Variants were marked as (a) “deleterious” based on MetaSVM annotations in dbNSFP and/or (b) “Personalis gene” variants based on whether they were in genes found in the Personalis Cancer Panel used in the Bartlett CTEP study.

A major goal of this exome sequencing evaluation was to identify responders/nonresponders from somatic mutations. To accomplish this, analyses were run with only “Personalis genes” and tested in the Bartlett CTEP dataset (a dataset generated using the Personalis ACE ExtendedCancer Panel on FL data). In both the more restricted (“Personalis genes”) and the full whole-exome datasets, statistical analyses were run on all likely somatic variants as well as only those gene variants inferred to be deleterious. Multiple classifiers using variable gene numbers were developed with nonresponder gene ranking based on a greedy algorithm (with a misclassification penalty) and responder/nonresponder binning using gene mutation status. Classification results were first assessed with 10-fold cross-validation within the dataset; subsequently, a subset of classifiers was assessed based on the overall ibrutinib response rate of the predicted responder group in the Bartlett CTEP FL subject study

### Summary of Variants

Exome data were generated from the paraffin-embedded tumor samples from 88 patients. 974,686 total nonsynonymous variants were identified. After filtering out potential errors and likely germline mutations, the number of variants was reduced to 13,554. Response data were available on 83 patients, comprising 17 responders and 66 nonresponders.

The final VAF histogram for filtered variants showed a significant reduction in the peaks at 0.5 and 1.0 seen in the original set of variants return by LabCorp, indicating a much higher ratio of somatic to germline variants. VAF values for EZH2-Y646 and STAT6-D419, known somatic FL-associated mutations, fell below 0.4, indicating that it would be reasonable to exclude variants not below this threshold if they were in dbSNP, but not in COSMIC. The variants in the dbSNP non-COSMIC set largely fell in the zones near 0.5 and 1.0, indicating that many of them are likely germline mutations. As a check of the final distribution of the filtered variants, the VAF distribution of the COSMIC (“known somatic”) variants found within the dataset was examined and found to have a similar distribution (note, however, that there are known contaminating variants in COSMIC that are likely to be nearly exclusively germline, accounting for the small peak around 0.5). The number of mutated genes in each sample varied from under 100 to over 500, and variance was greater across non-responder NR subjects, likely due to a larger sample size.

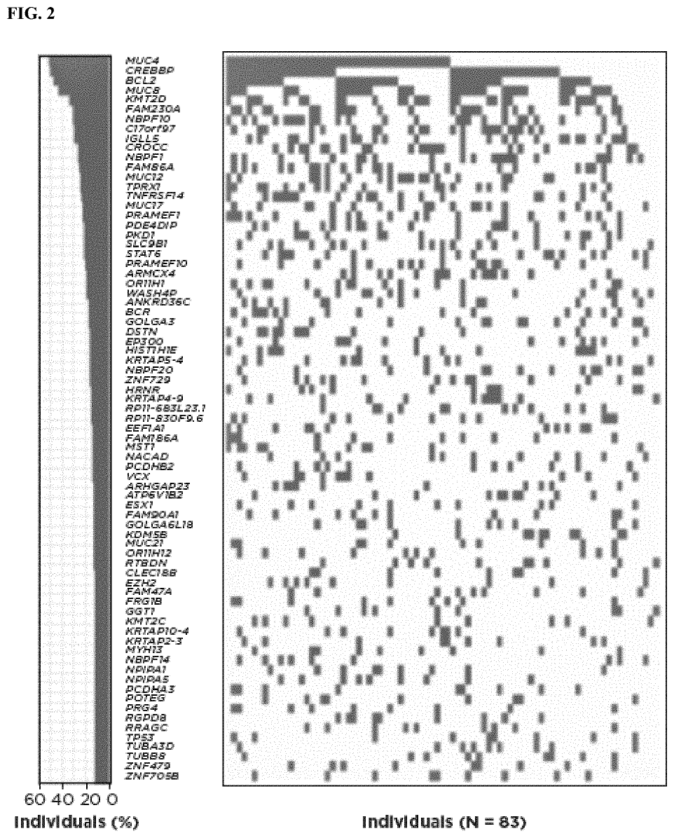

The overall pattern of variant frequencies identified from the whole exome sequencing is provided in FIG. 2. There were 75 genes with putative mutations in >10% of the patients, including many of those previously implicated in FL (e.g., CREBBP, BCL2, and KMT2D). The left panel of FIG. 2 shows the percentage of individuals with a mutation in each gene, while the right panel shows the distribution of mutations in those genes in the 83 patients for which responder data were available.

Due to the greater number of samples from nonresponders versus responders, univariate analysis yielded mostly variants significantly enriched in ibrutinib responders but in very low numbers, e.g., FANCA, HISTH1B, ANXA6, and PARP10 (Table 1). Interestingly, 2 patients with variants in BTG1, which is a tumor suppressor, also responded to ibrutinib. Few nonresponder genes were identified in univariate analysis, including NBPF1, ATP6AP1, EP400, and CNOT1 (mutations in these genes may activate pathways that bypass BTK, including the mTOR and JAK/STAT pathways).

TABLE 1
Univariate analysis of gene variants
in responders versus nonresponders*

| | Responder | Nonresponder | | |
|---|---|---|---|---|
| | (N = 17) | (N = 66) | Odds Ratio | p |
| Gene | n (%) | n (%) | (95% CI) | Value |
| FANCA | 3 (17.6) | 0 (0.0) | Inf (1.721-Inf) | 0.007 |
| HIST1H1B | 5 (29.4) | 3 (4.5) | 8.417 (1.426-61.654) | 0.008 |
| ANXA6 | 2 (11.8) | 0 (0.0) | Inf (0.750-Inf) | 0.04 |
| BTG1 | 2 (11.8) | 0 (0.0) | Inf (0.750-Inf) | 0.04 |
| DIAPH1 | 2 (11.8) | 0 (0.0) | Inf (0.750-Inf) | 0.04 |
| PARP10 | 2 (11.8) | 0 (0.0) | Inf (0.750-Inf) | 0.04 |
| PBRM1 | 2 (11.8) | 0 (0.0) | Inf (0.750-Inf) | 0.04 |
| PRDM1 | 2 (11.8) | 0 (0.0) | Inf (0.750-Inf) | 0.04 |
| RAD50 | 2 (11.8) | 0 (0.0) | Inf (0.750-Inf) | 0.04 |
| RECQL4 | 2 (11.8) | 0 (0.0) | Inf (0.750-Inf) | 0.04 |

Inf = infinite
*Results are shown only for genes with p values < 0.2.

### Cross-Validation Analysis

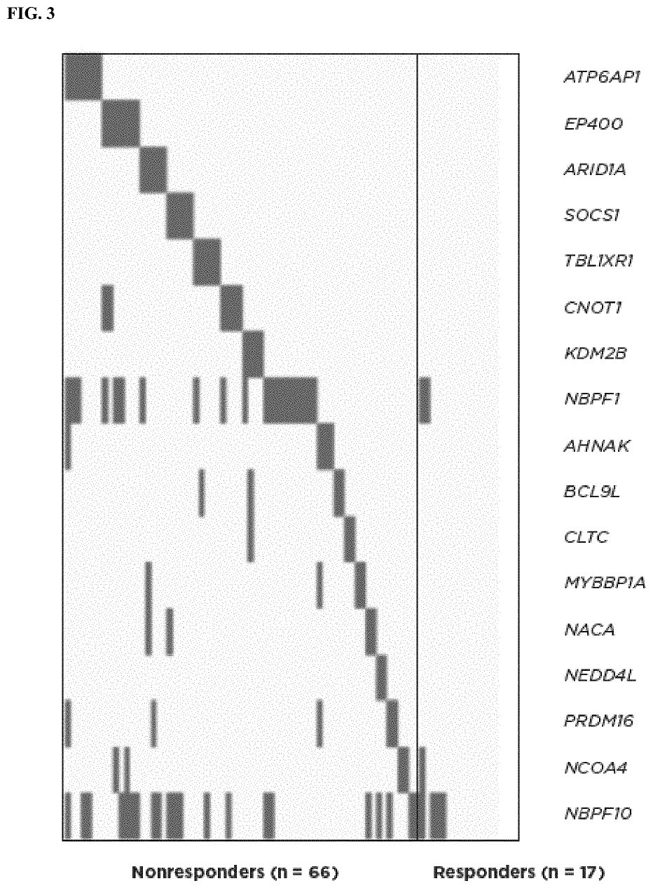

Because the genes that defined responders were few, genes mutated in more nonresponder patients were targeted for classifier development. A panel of genes were selected and ranked by choosing the gene that allowed inference of the most additional nonresponders in each iteration until all nonresponders were covered. From the selected panel, 17 classifier models were developed including variants in ATP6AP1, EP400, ARID1A, SOCS1, TBL1XR1, CNOT1, and KDM2B (FIG. 3).

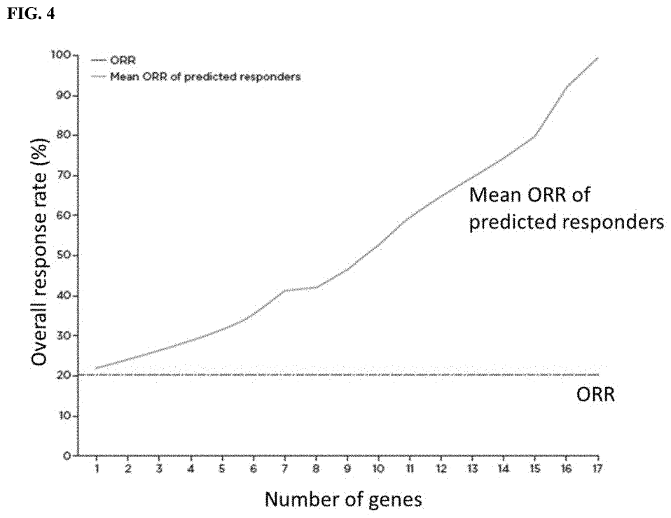

The mean ORR of predicted responders shown by the solid line (“mean ORR of predicted responders”) in FIG. 4 is based on 10-fold cross-validation for 17 different responder/nonresponder classification models, showing an increase in predicted ORR as more genes were added. Each model was defined by the number of genes used to build it, with genes being added in order of decreasing new information content, as shown in FIG. 3. The dotted line in FIG. 4 (“ORR”) represents the ORR of the entire patient cohort regardless of classification.

### Genes of Interest in Nonresponders

The mutation status of the top 5 ranked genes (ATP6AP1, EP400, ARID1A, SOCS1, and TBL1XR1) was most informative in predicting a lack of response. Mutations in these genes were found exclusively in nonresponders and are described below.

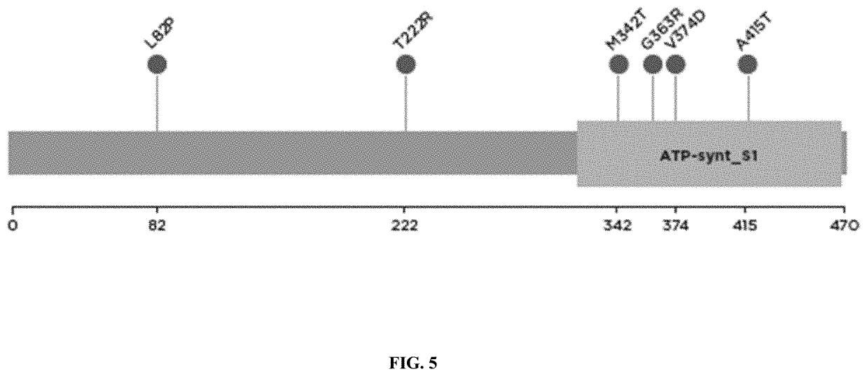

ATP6AP1—The majority of the mutations seen in the ATP6AP1 gene were found in the ATP-synthase S1 region (FIG. 5).

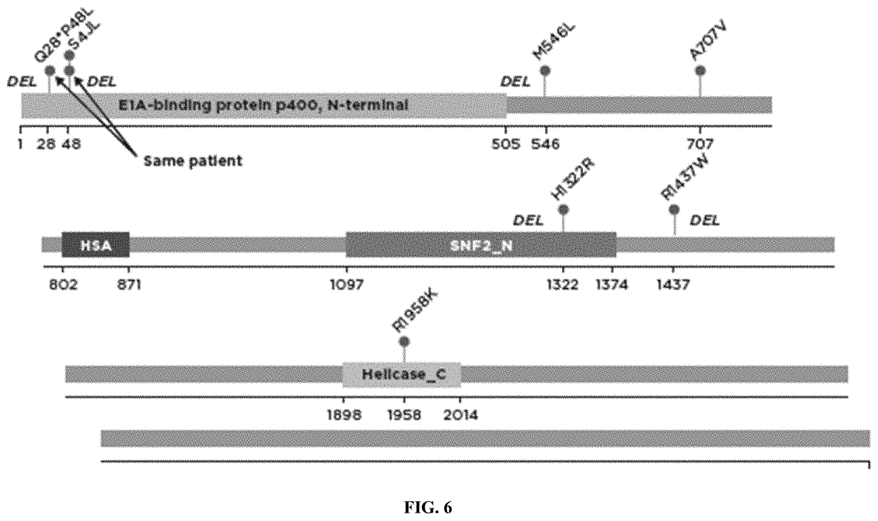

EP400—7 nonresponder patients had somatic mutations in the EP400 gene, and 5 of these patients had mutations marked as “deleterious” by metaSVM (FIG. 6). EP400 encodes a histone acetylase complex component.

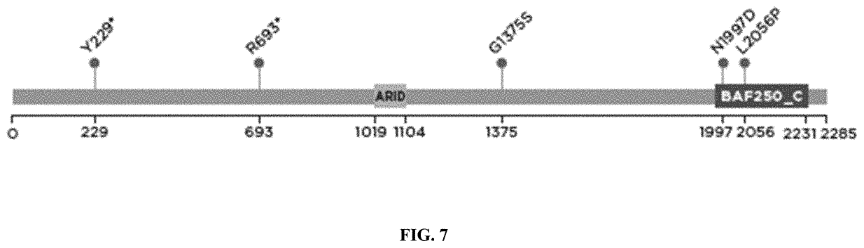

ARID1A—5 mutations in putative tumor suppressor ARID1A occurred in the DAWN dataset and 2 of these caused the formation of premature stop codons (FIG. 7).

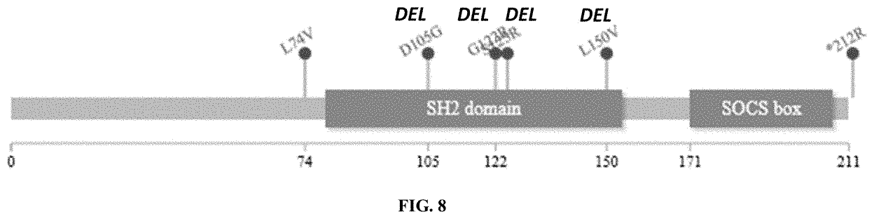

SOCS1—The majority of the 6 SOCS1 mutations observed in the DAWN study were predicted as deleterious by metaSVM and are in the SH2 domain (FIG. 8).

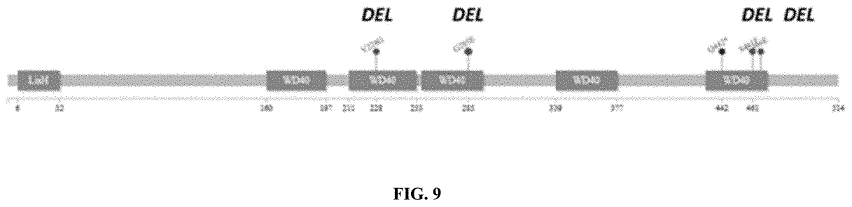

TBL1XR1—4 of the 5 putative somatic mutations in the TBLXR1 gene were predicted as deleterious by metaSVM; the remaining variant represents the gain of a premature stop codon (FIG. 9).

CARD11—CARD11 contained 8 variants found in 6 patients. Each of the CARD11 variants were identified individually, even though CARD11 was not a top ranked gene in this analysis. A total of 4 variants from 2 patients were left after the filtering applied here (T117P, D230N, C351S, and S352P), and could be deleterious, though they were not identified as deleterious by metaSVM. Of the variants filtered out, 1 had a variant allele frequency (VAF) of <0.05 (VAF=0.04672897), 1 was in the non-Catalogue Of Somatic Mutations In Cancer (COSMIC) dbSNP group that was subjected to the VAF<0.4 filter (VAF=0.49371981), and the others were marked in dbSNP as “germline only,” suggesting that much of the trend in CARD11 is due to germline variants.

### Somatic Mutations

Somatic mutations identified in the responders and nonresponders are provided in Table 2.

TABLE 2
Somatic mutations identified in DAWN FL patients

| | | | Codon | AA |
|---|---|---|---|---|
| Gene | Transcript | Allele | change | change |
| AHNAK | ENST00000378024 | A/T | gTt/gAt | V3640D |
| AHNAK | ENST00000378024 | T/C | aAg/aGg | K2180R |
| AHNAK | ENST00000378024 | C/T | Ggg/Agg | G799R |
| AHNAK | ENST00000378024 | G/C | Caa/Gaa | Q3796E |
| AHNAK | ENST00000378024 | T/C | gAt/gGt | D4045G |
| ANXA6 | ENST00000354546 | C/T | atG/atA | M582I |
| ANXA6 | ENST00000354546 | T/A | aaA/aaT | K374N |
| ARID1A | ENST00000324856 | C/T | Cga/Tga | R693* |
| ARID1A | ENST00000324856 | T/C | cTc/cCc | L2056P |
| ARID1A | ENST00000324856 | G/A | Ggc/Agc | G1375S |
| ARID1A | ENST00000324856 | A/G | Aat/Gat | N1997D |
| ARID1A | ENST00000324856 | C/A | taC/taA | Y229* |
| ATP6AP1 | ENST00000369762 | T/C | aTg/aCg | M342T |
| ATP6AP1 | ENST00000369762 | T/A | gTc/gAc | V374D |
| ATP6AP1 | ENST00000369762 | T/C | cTg/cCg | L82P |
| ATP6AP1 | ENST00000369762 | C/G | aCa/aGa | T222R |
| ATP6AP1 | ENST00000369762 | G/A | Gcc/Acc | A415T |
| ATP6AP1 | ENST00000369762 | G/A | Ggg/Agg | G363R |
| ATP6AP1 | ENST00000369762 | G/A | Ggg/Agg | G363R |
| BCL9L | ENST00000334801 | T/G | Aat/Cat | N627H |
| BCL9L | ENST00000334801 | T/G | Aat/Cat | N627H |
| BCL9L | ENST00000334801 | T/G | Aat/Cat | N627H |
| BCL9L | ENST00000334801 | T/G | Aat/Cat | N627H |
| BTG1 | ENST00000256015 | G/C | Cga/Gga | R35G |
| BTG1 | ENST00000256015 | C/T | Gaa/Aaa | E50K |
| CLTC | ENST00000269122 | C/A | aCc/aAc | T109N |
| CLTC | ENST00000269122 | G/C | Gca/Cca | A81P |
| CLTC | ENST00000269122 | G/A | Gca/Aca | A68T |
| CNOT1 | ENST00000317147 | T/C | Aca/Gca | T818A |
| CNOT1 | ENST00000317147 | T/A | gAt/gTt | D2179V |
| CNOT1 | ENST00000317147 | C/T | gGc/gAc | G404D |
| CNOT1 | ENST00000317147 | C/T | gGa/gAa | G690E |
| CNOT1 | ENST00000317147 | G/A | cCa/cTa | P1628L |
| CNOT1 | ENST00000317147 | G/A | aCt/aTt | T927I |
| DIAPH1 | ENST00000253811 | G/A | Ctc/Ttc | L977F |
| DIAPH1 | ENST00000253811 | C/A | Gtt/Ttt | V762F |
| EP400 | ENST00000333577 | C/T | Cgg/Tgg | R1437W |
| EP400 | ENST00000333577 | A/C | Atg/Ctg | M546L |
| EP400 | ENST00000333577 | C/T | gCg/gTg | A707V |
| EP400 | ENST00000333577 | C/T | Cag/Tag | Q28* |
| EP400 | ENST00000333577 | C/T | tCg/tTg | S49L |
| EP400 | ENST00000333577 | A/G | cAt/cGt | H1322R |
| EP400 | ENST00000333577 | G/A | aGg/aAg | R1958K |
| EP400 | ENST00000333577 | C/T | cCg/cTg | P48L |
| FANCA | ENST00000389301 | G/A | Cgc/Tgc | R1011C |
| FANCA | ENST00000389301 | C/A | agG/agT | R1349S |
| FANCA | ENST00000389301 | A/G | cTg/cCg | L379P |
| HIST1H1B | ENST00000331442 | C/T | gGc/gAc | G106D |
| HIST1H1B | ENST00000331442 | C/T | gGc/gAc | G73D |
| HIST1H1B | ENST00000331442 | C/T | Gct/Act | A215T |
| HIST1H1B | ENST00000331442 | C/G | ttG/ttC | L90F |
| HIST1H1B | ENST00000331442 | C/T | Gct/Act | A215T |
| HIST1H1B | ENST00000331442 | G/T | agC/agA | S89R |
| HIST1H1B | ENST00000331442 | C/G | Gct/Cct | A50P |
| HIST1H1B | ENST00000331442 | C/G | Gct/Cct | A131P |
| HIST1H1B | ENST00000331442 | C/G | aGc/aCc | S89T |
| HIST1H1B | ENST00000331442 | C/T | gGc/gAc | G106D |
| KDM2B | ENST00000377071 | C/T | Gat/Aat | D122N |
| KDM2B | ENST00000377071 | C/T | Gcc/Acc | A818T |
| KDM2B | ENST00000377071 | G/A | Cgg/Tgg | R1025W |
| KDM2B | ENST00000377071 | T/A | Aca/Tca | T602S |
| MYBBP1A | ENST00000381556 | G/C | Ctg/Gtg | L1323V |
| MYBBP1A | ENST00000381556 | G/A | Cgt/Tgt | R885C |
| MYBBP1A | ENST00000381556 | G/C | cCg/cGg | P500R |
| MYBBP1A | ENST00000381556 | C/A | Gac/Tac | D749Y |
| NACA | ENST00000454682 | A/G | Tcc/Ccc | S1190P |
| NACA | ENST00000454682 | A/G | Tcc/Ccc | S1190P |
| NACA | ENST00000454682 | C/G | aGc/aCc | S394T |
| NACA | ENST00000454682 | A/G | Tcc/Ccc | S1190P |
| NBPF1 | ENST00000430580 | C/A | gGt/gTt | G916V |
| NBPF1 | ENST00000430580 | G/A | cCg/cTg | P675L |
| NBPF1 | ENST00000430580 | T/A | cAg/cTg | Q1132L |
| NBPF1 | ENST00000430580 | T/A | cAg/cTg | Q1132L |
| NBPF1 | ENST00000430580 | T/A | aAg/aTg | K623M |
| NBPF1 | ENST00000430580 | T/C | Agc/Ggc | S853G |
| NBPF1 | ENST00000430580 | C/G | caG/caC | Q650H |
| NBPF1 | ENST00000430580 | C/T | tGc/tAc | C663Y |
| NBPF1 | ENST00000430580 | A/G | Tgt/Cgt | C251R |
| NBPF1 | ENST00000430580 | T/A | Agg/Tgg | R364W |
| NBPF1 | ENST00000430580 | T/C | gAa/gGa | E1011G |
| NBPF1 | ENST00000430580 | G/T | gaC/gaA | D905E |
| NBPF1 | ENST00000430580 | T/A | gAt/gTt | D896V |
| NBPF1 | ENST00000430580 | C/T | Gag/Aag | E448K |
| NBPF1 | ENST00000430580 | C/T | Ggc/Agc | G6S |
| NBPF1 | ENST00000430580 | T/G | Aag/Cag | K59Q |
| NBPF1 | ENST00000430580 | T/A | aAg/aTg | K623M |
| NBPF1 | ENST00000430580 | T/A | cAg/cTg | Q1132L |
| NBPF1 | ENST00000430580 | C/G | caG/caC | Q650H |
| NBPF1 | ENST00000430580 | C/A | Gtt/Ttt | V174F |
| NBPF1 | ENST00000430580 | C/A | Gtg/Ttg | V1100L |
| NBPF1 | ENST00000430580 | G/A | tCt/tTt | S611F |
| NBPF1 | ENST00000430580 | C/T | Gaa/Aaa | E439K |
| NBPF1 | ENST00000430580 | T/A | aAg/aTg | K623M |
| NBPF1 | ENST00000430580 | G/C | Cct/Gct | P1070A |
| NBPF1 | ENST00000430580 | C/T | Ggc/Agc | G1062S |
| NBPF1 | ENST00000430580 | C/G | caG/caC | Q650H |
| NBPF1 | ENST00000430580 | C/G | caG/caC | Q650H |
| NBPF1 | ENST00000430580 | T/A | aAg/aTg | K623M |
| NBPF1 | ENST00000430580 | C/T | atG/atA | M1133I |
| NBPF1 | ENST00000430580 | T/A | aAg/aTg | K623M |
| NBPF1 | ENST00000430580 | T/A | Agg/Tgg | R364W |
| NBPF1 | ENST00000430580 | C/G | caG/caC | Q650H |
| NBPF10 | ENST00000342960 | G/C | aaG/aaC | K56N |
| NBPF10 | ENST00000342960 | C/T | Ccc/Tcc | P1180S |
| NBPF10 | ENST00000342960 | C/T | Ctc/Ttc | L92F |
| NBPF10 | ENST00000342960 | A/G | gAg/gGg | E1171G |
| NBPF10 | ENST00000342960 | G/T | Ggg/Tgg | G387W |
| NBPF10 | ENST00000342960 | C/T | Ctc/Ttc | L92F |
| NBPF10 | ENST00000342960 | C/A | tgC/tgA | C1179* |
| NBPF10 | ENST00000342960 | A/T | cAg/cTg | Q3488L |
| NBPF10 | ENST00000342960 | A/G | gAc/gGc | D65G |
| NBPF10 | ENST00000342960 | C/T | Ctc/Ttc | L92F |
| NBPF10 | ENST00000342960 | C/T | Ctc/Ttc | L92F |
| NBPF10 | ENST00000342960 | C/T | Ctc/Ttc | L92F |
| NBPF10 | ENST00000342960 | C/T | Ctc/Ttc | L92F |
| NBPF10 | ENST00000342960 | C/T | Ctc/Ttc | L92F |
| NBPF10 | ENST00000342960 | C/T | Ctc/Ttc | L92F |
| NBPF10 | ENST00000342960 | C/A | aaC/aaA | N308K |
| NBPF10 | ENST00000342960 | C/T | Cga/Tga | R104* |
| NBPF10 | ENST00000342960 | C/T | Ctc/Ttc | L92F |
| NBPF10 | ENST00000342960 | C/T | Cga/Tga | R375* |
| NBPF10 | ENST00000342960 | A/T | gaA/gaT | E3455D |
| NBPF10 | ENST00000342960 | C/T | Ctc/Ttc | L92F |
| NBPF10 | ENST00000342960 | C/T | Ctc/Ttc | L92F |
| NBPF10 | ENST00000342960 | G/A | cGc/cAc | R25H |
| NBPF10 | ENST00000342960 | G/T | Gcc/Tcc | A48S |
| NBPF10 | ENST00000342960 | G/C | caG/caC | Q142H |
| NBPF10 | ENST00000342960 | C/T | Ctc/Ttc | L92F |
| NBPF10 | ENST00000342960 | C/T | Ctc/Ttc | L92F |
| NCOA4 | ENST00000452682 | A/C | gaA/gaC | E54D |
| NCOA4 | ENST00000431200 | G/A | Gca/Aca | A7T |
| NCOA4 | ENST00000431200 | G/A | Gca/Aca | A7T |
| NCOA4 | ENST00000431200 | T/G | cTa/cGa | L9R |
| NCOA4 | ENST00000452682 | G/A | cGg/cAg | R52Q |
| NCOA4 | ENST00000431200 | G/A | Gca/Aca | A7T |
| NEDD4L | ENST00000400345 | G/A | Gag/Aag | E271K |
| NEDD4L | ENST00000400345 | C/T | Cag/Tag | Q305* |
| PARP10 | ENST00000525773 | C/T | Gac/Aac | D260N |
| PARP10 | ENST00000525773 | C/T | Gag/Aag | E1017K |
| PBRM1 | ENST00000296302 | G/T | cCt/cAt | P1343H |
| PBRM1 | ENST00000296302 | G/T | gaC/gaA | D159E |
| PRDM1 | ENST00000369096 | T/C | aTt/aCt | I329T |
| PRDM1 | ENST00000369096 | G/A | Gtg/Atg | V250M |
| PRDM16 | ENST00000270722 | C/T | Ccc/Tcc | P112S |
| PRDM16 | ENST00000270722 | G/A | Gtg/Atg | V48M |
| PRDM16 | ENST00000270722 | C/A | cCa/cAa | P50Q |
| PRDM16 | ENST00000270722 | C/T | aCc/aTc | T609I |
| PRDM16 | ENST00000270722 | A/G | Aat/Gat | N161D |
| RAD50 | ENST00000434288 | C/A | taC/taA | Y109* |
| RAD50 | ENST00000265335 | A/G | aAa/aGa | K398R |
| RAD50 | ENST00000265335 | C/T | Cga/Tga | R365* |
| RECQL4 | ENST00000428558 | C/A | cGg/cTg | R755L |
| RECQL4 | ENST00000428558 | C/T | Gga/Aga | G892R |
| SOCS1 | ENST00000332029 | G/T | agC/agA | S125R |
| SOCS1 | ENST00000332029 | T/C | gAc/gGc | D105G |
| SOCS1 | ENST00000332029 | A/T | Tga/Aga | 212R** |
| SOCS1 | ENST00000332029 | G/C | Ctg/Gtg | L74V |
| SOCS1 | ENST00000332029 | C/T | Gga/Aga | G122R |
| SOCS1 | ENST00000332029 | G/C | Ctg/Gtg | L150V |
| TBL1XR1 | ENST00000430069 | G/A | Caa/Taa | Q442* |
| TBL1XR1 | ENST00000430069 | A/C | gTc/gGc | V228G |
| TBL1XR1 | ENST00000430069 | G/A | tCt/tTt | S461F |
| TBL1XR1 | ENST00000430069 | C/T | gGa/gAa | G285E |
| TBL1XR1 | ENST00000430069 | C/T | gGa/gAa | G285E |
| TBL1XR1 | ENST00000430069 | A/T | gTa/gAa | V466E |

* Stop codon gained; ** Stop codon lost.

### CONCLUSIONS

Mutational analysis of genes in patients from the phase 2 DAWN trial yielded insights into the mechanism of ibrutinib response and resistance in R/R FL.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed:

1. A method of treating follicular lymphoma (FL) in a subject, the method comprising:
administering to the subject a therapeutically effective amount of ibrutinib to thereby treat the FL, wherein the subject does not have one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1.

2. A method of treating follicular lymphoma (FL) in a subject not having one or more mutations as defined in Table 2 in one or more genes selected from AHNAK, ARID1A, ATP6AP1, BCL9L, CLTC, CNOT1, EP400, KDM2B, MYBBPA, NACA, NBPF1, NBPF10, NCOA4, NEDD4L, PRDM16, SOCS1, and TBL1XR1, the method comprising:
administering to the subject a therapeutically effective amount of ibrutinib to thereby treat the FL.

3. The method of claim 1, wherein the therapeutically effective amount of ibrutinib comprises from about 420 mg to about 840 mg.

4. The method of claim 3, wherein the therapeutically effective amount of ibrutinib comprises 560 mg.

5. The method of claim 1, wherein the FL is relapsed/refractory (R/R) FL.

6. The method of claim 1, wherein, prior to the administering, the subject had a diagnosis of grade 1, 2, or 3a nontransformed FL.

7. The method of claim 6, wherein, prior to the administering, the subject had been treated with >2 prior lines of therapy.

8. The method of claim 7, wherein, prior to the administering, the subject was R/R to a last prior line of therapy with an anti-CD20 monoclonal antibody-containing chemoimmunotherapy regimen.

9. The method of claim 1, wherein the subject has a partial response or a complete response.

10. A method treating follicular lymphoma (FL) in a subject, the method comprising:
analyzing a sample from the subject for one or more mutations as defined in Table 2 in one or more genes selected from AHNAK, ARID1A, ATP6AP1, BCL9L, CLTC, CNOT1, EP400, KDM2B, MYBBP1A, NACA, NBPF1, NBPF10, NCOA4, NEDD4L, PRDM16, SOCS1, and TBL1XR1, wherein the one or more mutations in the one or more genes is indicative of nonresponsiveness to ibrutinib; and
administering a therapeutically effective amount of ibrutinib to thereby treat the FL.
